(19) United States
(12) Patent Application Publication
(10) Pub. No.: US 2013/0211230 A1
(43) Pub. Date: Aug. 15, 2013

(54) SYSTEM AND METHOD FOR USING MEDICAL IMAGE FUSION

(71) Applicant: Convergent Life Sciences, Inc., (US)

(72) Inventor: DANIEL S. SPERLING, West Orange, NJ (US)

(73) Assignee: Convergent Life Sciences, Inc., Los Angeles, CA (US)

(21) Appl. No.: 13/762,475

(22) Filed: Feb. 8, 2013

Related U.S. Application Data
(60) Provisional application No. 61/596,372, filed on Feb. 8, 2012.

(51) Int. Cl. A61B 8/08; A61B 8/13; A61B 6/03; A61B 5/055

(52) U.S. Cl. CPC A61B 8/5261; A61B 5/055; A61B 8/13; A61B 6/037; A61B 6/032; A61B 8/085
USPC 600/410; 600/425

(57) ABSTRACT

A method and system for diagnosis and treatment of medical conditions. The method includes communicating MRI, CT, PET and/or ultrasound image data, and fusing such data using an image-guided biopsy system. It further includes using such fused images in conjunction with the image-guided biopsy system for performing diagnosis and treatment procedures.

### CROSS REFERENCE TO RELATED APPLICATIONS

The present application is a non-provisional of U.S. Provisional Patent Application 61/596,372, filed Feb. 9, 2012, the entirety of which is expressly incorporated herein by reference.

### BACKGROUND OF THE INVENTION

1. Field of the Invention

The present disclosure relates to medical imaging and surgical procedures.

2. Description of the Art

Prostate cancer is one of the most common types of cancer affecting men. It is a slow growing cancer, which is easily treatable if identified at an early stage. A prostate cancer diagnosis often leads to surgery or radiation therapy. Such treatments are costly and can cause serious side effects, including incontinence and erectile dysfunction. Unlike many other types of cancer, prostate cancer is not always lethal and often is unlikely to spread or cause harm. Many patients who are diagnosed with prostate cancer receive radical treatment even though it would not prolong the patient's life, ease pain, or significantly increase the patient's health.

Prostate cancer may be diagnosed by taking a biopsy of the prostate, which is conventionally conducted under the guidance of ultrasound imaging. Ultrasound imaging has high spatial resolution, and is relatively inexpensive and portable. However, ultrasound imaging has relatively low tissue discrimination ability. Accordingly, ultrasound imaging provides adequate imaging of the prostate organ, but it does not provide adequate imaging of tumors within the organ due to the similarity of cancer tissue and benign tissues, as well as the lack of tissue uniformity. Due to the inability to visualize the cancerous portions within the organ with ultrasound, the entire prostate must be considered during the biopsy. Thus, in the conventional prostate biopsy procedure, a urologist relies on the guidance of two-dimensional ultrasound to systematically remove tissue samples from various areas throughout the entire prostate, including areas that are free from cancer.

Magnetic Resonance Imaging (MRI) has long been used to evaluate the prostate and surrounding structures. MRI is in some ways superior to ultrasound imaging because it has very good soft tissue contrast. There are several types of MRI techniques, including T2 weighted imaging, diffusion weighted imaging, and dynamic contrast imaging. Standard T2-weighted imaging does not discriminate cancer from other processes with acceptable accuracy. Diffusion-weighted imaging and dynamic contrast imaging may be integrated with traditional T2-weighted imaging to produce multi-parametric MRI. The use of multi-parametric MRI has been shown to improve sensitivity over any single parameter and may enhance overall accuracy in cancer diagnosis.

As with ultrasound imaging, MRI also has limitations. For instance, it has a relatively long imaging time, requires specialized and costly facilities, and is not well-suited for performance by a urologist at a urology center. Furthermore, performing direct prostate biopsy within MRI machines is not practical for a urologist at a urology center.

To overcome these shortcomings and maximize the usefulness of the MRI and ultrasound imaging modalities, methods and devices have been developed for digitizing medical images generated by multiple imaging modalities (e.g., ultrasound and MRI) and fusing or integrating multiple images to form a single composite image. This composite image includes information from each of the original images that were fused together. A fusion or integration of Magnetic Resonance (MR) images with ultrasound-generated images has been useful in the analysis of prostate cancer within a patient. Image-guided biopsy systems, such as the Artemis produced by Eigen, and UroStation developed by Koelis, have been invented to aid in fusing MRI and ultrasonic modalities. These systems are three-dimensional (3D) image-guided prostate biopsy systems that provide tracking of biopsy sites within the prostate.

Until now, however, such systems have not been adequate for enabling MRI-ultrasound fusion to be performed by a urologist at a urology center. The use of such systems for MRI-ultrasound fusion necessarily requires specific MRI data, including MRI scans, data related to the assessment of those scans, and data produced by the manipulation of such data. Such MRI data, however, is not readily available to urologists and it would be commercially impractical for such MRI data to be generated at a urology center. This is due to many reasons, including urologists' lack of training or expertise, as well as the lack of time, to do so. Also, it is uncertain whether a urologist can profitably implement an image-guided biopsy system in his or her practice while contemporaneously attempting to learn to perform MRI scans. Furthermore, even if a urologist invested the time and money in purchasing MRI equipment and learning to perform MRI scans, the urologist would still be unable to perform the MRI-ultrasound fusion because a radiologist is needed for the performance of advanced MRI assessment and manipulation techniques which are outside the scope of a urologist's expertise.

MRI is generally considered to offer the best soft tissue contrast of all imaging modalities. Both anatomical (e.g., T₁, T₂) and functional MRI, e.g. dynamic contrast-enhanced (DCE), magnetic resonance spectroscopic imaging (MRSI) and diffusion-weighted imaging (DWI) can help visualize and quantify regions of the prostate based on specific attributes. Zonal structures within the gland cannot be visualized clearly on T₁ images. However a hemorrhage can appear as high-signal intensity after a biopsy to distinguish normal and pathologic tissue. In T₂ images, zone boundaries can be easily observed. Peripheral zone appears higher in intensity relative to the central and transition zone. Cancers in the peripheral zone are characterized by their lower signal intensity compared to neighboring regions. DCE improves specificity over T₂ imaging in detecting cancer. It measures the vascularity of tissue based on the flow of blood and permeability of vessels. Tumors can be detected based on their early enhancement and early washout of the contrast agent. DWI measures the water diffusion in tissues. Increased cellular density in tumors reduces the signal intensity on apparent diffusion maps.

The use of imaging modalities other than trans-rectal ultrasound (TRUS) for biopsy and/or therapy typically provides a number of logistic problems. For instance, directly using MRI to navigate during biopsy or therapy can be complicated (e.g. requiring use of nonmagnetic materials) and expensive (e.g., MRI operating costs). This need for specially designed tracking equipment, access to an MRI machine, and limited availability of machine time has resulted in very limited use of direct MRI-guided biopsy or therapy. CT imaging is likewise expensive and has limited access, and poses a radiation risk for operators and patient.

Accordingly, one known solution is to register a pre-acquired image (e.g., an MRI or CT image), with a 3D TRUS image acquired during a procedure. Regions of interest identifiable in the pre-acquired image volume may be tied to corresponding locations within the TRUS image such that they may be visualized during/prior to biopsy target planning or therapeutic application. This solution allows a radiologist to acquire, analyze and annotate MRI/CT scan at the image acquisition facility while a urologist can still perform the procedure using live ultrasound in his/her clinic.

Consequently, there exists a need for a method and system for facilitating the storage, communication, and implementation of image data between multiple medical centers to enable MRI-ultrasound fusion to be performed at a urology center.

### SUMMARY

The phrase “image fusion” is sometimes used to define the process of registering two images that are acquired via different imaging modalities or at different time instances. The registration/fusion of images obtained from different modalities creates a number of complications. The shape of soft tissues in two images may change between acquisitions of each image. Likewise, a diagnostic or therapeutic procedure can alter the shape of the object that was previously imaged. Further, in the case of prostate imaging the frame of reference (FOR) of the acquired images is typically different. That is, multiple MRI volumes are obtained in high resolution transverse, coronal or sagittal planes respectively, with lower resolution representing the slice distance. These planes are usually in rough alignment with the patient's head-toe, anterior-posterior or left-right orientations. In contrast, TRUS images are often acquired while a patient lays on his side in a fetal position by reconstructing multiple rotated samples 2D frames to a 3D volume. The 2D image frames are obtained at various instances of rotation of the TRUS probe after insertion in to the rectal canal. The probe is inserted at an angle (approximately 30-45 degrees) to the patient's head-toe orientation. As a result the gland in MRI and TRUS will need to be rigidly aligned because their relative orientations are unknown at scan time. Typically, well-defined and invariant anatomical landmarks may be used to register the images, though since the margins of landmarks themselves vary with imaging modality, the registration may be imperfect or require discretion in interpretation. A further difficulty with these different modalities is that the intensity of objects in the images do not necessarily correspond. For instance, structures that appear bright in one modality (e.g., MRI) may appear dark in another modality (e.g., ultrasound). Thus, the logistical process of overlaying or merging the images requires perceptual optimization. In addition, structures identified in one image (soft tissue in MRI) may be entirely absent in another image. TRUS imaging causes further deformation of gland due to pressure exerted by the TRUS transducer on prostate. As a result, rigid registration is not sufficient to account for difference between MRI and TRUS images. Finally, the resolution of the images may also impact registration quality.

Due to the FOR differences, image intensity differences between MRI and TRUS images, and/or the potential for the prostate to change shape between imaging by the MRI and TRUS scans, one of the few known correspondences between the prostate images acquired by MRI and TRUS is the boundary/surface model of the prostate. That is, the prostate is an elastic object that has a gland boundary or surface model that defines the volume of the prostate. By defining the gland surface boundary in the dataset for each modality, the boundary can then be used as a reference for aligning both images. Thus, each point of the volume defined within the gland boundary of the prostate in one image should correspond to a point within a volume defined by a gland boundary of the prostate in the other image, and vice versa. In seeking to register the surfaces, the data in each data set may be transformed, assuming elastic deformation of the prostate gland.

According to a first aspect, a system and method is provided for use in medical imaging of a prostate of a patient. The utility includes obtaining a first 3D image volume from an MRI imaging device. Typically, this first 3D image volume is acquired from data storage. That is, the first 3D image volume is acquired at a time prior to a current procedure. A first shape or surface model may be obtained from the MRI image (e.g., a triangulated mesh describing the gland). The surface model can be manually or automatically extracted from all co-registered MRI image modalities. That is, multiple MRI images may themselves be registered with each other as a first step. The 3D image processing may be automated, so that a technician need not be solely occupied by the image processing, which may take seconds or minutes. The MRI images may be T₁, T₂, DCE (dynamic contrast-enhanced), DWI (diffusion weighted imaging), ADC (apparent diffusion coefficient) or other.

Similarly, data from other imaging modalities, e.g., computer aided tomography (CAT) scans can also be registered. In the case of a CAT scan, the surface of the prostate may not represent a high contrast feature, and therefore other aspects of the image may be used; typically, the CAT scan is used to identify radiodense features, such as calcifications, or brachytherapy seeds, and therefore the goal of the image registration process would be to ensure that these features are accurately located in the fused image model. A co-registered CT image with PET scan can also provide diagnostic information that can be mapped to TRUS frame of reference for image guidance.

An ultrasound volume of the patient's prostate is then obtained, for example, through rotation of the TRUS probe, and the gland boundary is segmented in the ultrasound image. The ultrasound images acquired at various angular positions of the TRUS probe during rotation can be reconstructed to a rectangular grid uniformly through intensity interpolation to generate a 3D TRUS volume. Of course, other ultrasound methods may be employed without departing from the scope of the technology. The MRI or CAT scan volume is registered to the 3D TRUS volume (or vice versa), and a registered image of the 3D TRUS volume is generated in the same frame of reference (FOR) as the MRI or CAT scan image. According to a preferred aspect, this registration occurs prior to a diagnostic or therapeutic intervention. The advantage here is that both data sets may be fully processed, with the registration of the 3D TRUS volume information completed. Thus, during a later real-time TRUS guided diagnostic or therapeutic procedure, a fully fused volume model is available. In general, the deviation of a prior 3D TRUS scan from a subsequent one will be small, so features from the real-time scan can be aligned with those of the prior imaging procedure. The fused image from the MRI (or CAT) scan provides better localization of the suspect pathological tissue, and therefore guidance of the diagnostic biopsy or therapeutic intervention. Therefore, the suspect voxels from the MRI are highlighted in the TRUS image, which during a procedure would be presented in 2D on a display screen to guide the urologist. The process therefore seeks to register 3 sets of data; the MRI (or other scan) information, the pre-operative 3D TRUS information, and the real time TRUS used during the procedure. Ideally, the preoperative 3D TRUS and the interoperative TRUS are identical apparatus, and therefore would provide maximum similarity and either minimization of artifacts or present the same artifacts. Indeed, the 3D TRUS preoperative scan can be obtained using the same TRUS scanner and immediately pre-operative, though it is preferred that the registration of the images proceed under the expertise of a radiologist or medical scanning technician, who may not be immediately available during that period.

The registered image and the geometric transformation that relates the MRI scan volume with the ultrasound volume can be used to guide a medical procedure such as, for example, biopsy or brachytherapy.

These regions of interest identified on the MRI scan are usually defined by a radiologist based on information available in MRI prior to biopsy, and may be a few points, point clouds representing regions, or triangulated meshes. Likewise, the 3D TRUS may also reveal features of interest for biopsy, which may also be marked as regions of interest. Because of the importance of registration of the regions of interest in the MRI scan with the TRUS used intraoperatively, the radiologist can override or control the image fusion process according to his or her discretion.

Segmented MRI and 3D TRUS is obtained from a patient for the prostate grand. The MRI and TRUS data is registered and transformations applied to form a fused image in which voxels of the MRI and TRUS images physically correspond to one another. Regions of interest are then identified either from the source images or from the fused image. The regions of interest are then communicated to the real-time ultrasound system, which tracks the earlier TRUS image. Because the ultrasound image is used for real time guidance, typically the transformation/alignment takes place on the MRI data, which can then be superposed or integrated with the ultrasound data.

During the procedure, the real-time TRUS display is supplemented with the MRI (or CAT or other scan) data, and an integrated display presented to the urologist. In some cases, haptic feedback may be provided so that the urologist can “feel” features when using a tracker.

It is noted that as an alternate, the MRI or CAT scan data may be used to provide a coordinate frame of reference for the procedure, and the TRUS image modified in real-time to reflect an inverse of the ultrasound distortion. That is, the MRI or CAT data typically has a precise and undistorted geometry. On the other hand the ultrasound image may be geometrically distorted by phase velocity variations in the propagation of the ultrasound waves through the tissues, and to a lesser extent, by reflections and resonances. Since the biopsy instrument itself is rigid, it will correspond more closely to the MRI or CAT model than the TRUS model, and therefore a urologist seeking to acquire a biopsy sample may have to make corrections in course if guided by the TRUS image. If the TRUS image, on the other hand, is normalized to the MRI coordinate system, then such corrections may be minimized. This requires that the TRUS data be modified according to the fused image volume model in real time. However, modern graphics processors (GPU or APU, multicore CPU, FPGA) and other computing technologies make this possible.

According to another aspect, the urologist is presented with a 3D display of the patient's anatomy, supplemented by and registered to the real-time TRUS data. Such 3D displays are effectively used with haptic feedback.

It is noted that two different image transformations are at play; the first is a frame of reference transformation, due to the fact that the MRI image is created as a set of slices in parallel planes which will generally differ from the image plane of the TRUS, defined by the probe angle. The second transformation represents the elastic deformation of the objects within the image to properly aligned surfaces and landmarks.

It is therefore an object to provide a method for guiding a procedure, comprising: annotating regions of a medical imaging scan to acquire a first image of an organ; modeling the medical imaging scan as an imaging scan volumetric model; communicating the annotations of the medical imaging scan and the volumetric model through a communication network to an ultrasound center; processing ultrasound data from an ultrasound scanner at the ultrasound center to form an ultrasound volumetric model of the organ; fusing the medical imaging volumetric model with the ultrasound volumetric model into a fused image based on predetermined anatomical features, wherein at least one of the medical imaging volumetric model and the ultrasound volumetric model is deformed according to a tissue model such that the predetermined anatomical features of the medical imaging volumetric model and the ultrasound volumetric model are aligned; and merging real-time ultrasound data with the fused image and annotated regions at the ultrasound center, such that that the annotated regions of the medical imaging scan are presented on a display maintaining anatomically accurate relationships with the real-time ultrasound data.

It is also an object to provide a system for guiding a procedure, comprising: a memory configured to store annotated regions of a medical imaging scan of an organ; a memory configured to store a model of the medical imaging scan as an imaging scan volumetric model;

a communication port configured to communicate the stored annotated regions and the model through a communication network; at least one processor configured to form an ultrasound volumetric model of the organ from ultrasound data, to fuse the communicated model with the ultrasound volumetric model based on predetermined anatomical features, wherein at least one of the communicated model and the ultrasound volumetric model is deformed according to a tissue model such that the predetermined anatomical features of the communicated model and the ultrasound volumetric model are aligned; and a real-time ultrasound system configured to merge real-time ultrasound data with the fused communicated model and ultrasound volumetric model, and to present the annotated regions on a display maintaining anatomically accurate relationships with the real-time ultrasound data.

It is a still further object to provide a system for guiding a procedure, comprising: a communication port configured to receive information defining a three dimensional volumetric model of an organ synthesized from a plurality of slices, and annotations of portions of the three dimensional volumetric model; at least one processor configured to: form an ultrasound volumetric model of the organ from ultrasound planar scans, define anatomical landmarks in the ultrasound volumetric model; define tissue deformation properties of tissues represented in the ultrasound volumetric model; fuse the communicated three dimensional volumetric model with the ultrasound volumetric model to form a fused model, based on at least the defined anatomical features and the defined tissue deformation properties, such that the predetermined anatomical features of the three dimensional volumetric model and the ultrasound volumetric model are aligned; and a real-time ultrasound system configured to display real-time ultrasound data with at least the annotations of the portions of the three dimensional volumetric model superimposed in anatomically accurate positions.

The modeling may comprise a segmentation of anatomical features.

The method may further comprise transforming at least one of the imaging scan volumetric model and the ultrasound volumetric model to a common physical coordinate system such that the common anatomy of the organ is in a corresponding coordinate position. The system may further comprise at least one transform processor configured to transform at least one of the imaging scan volumetric model and the ultrasound volumetric model to a common physical coordinate system, such that the common anatomy of the organ is in a corresponding coordinate position.

A projection of the defined features in the common physical coordinate system may be projected into a native coordinate system of the real-time ultrasound data. The at least one transform processor may be configured to determine a projection of the defined features in the common physical coordinate system into a native coordinate system of the real-time ultrasound data.

The medical imaging scan may comprise a magnetic resonance imaging scan and/or a computed aided tomography imaging scan.

The organ may comprise a prostate gland. The predetermined anatomical features may comprise at least one portion of a urethra.

The medical imaging scan may comprise a magnetic resonance imaging scan having plurality of magnetic resonance planar images displaced along an axis, and the ultrasound data may comprise a plurality of ultrasound planar images, wherein the plurality of magnetic resonance planar images are inclines with respect to the plurality of ultrasound planar images.

The annotated regions may be superimposed on the display of the real-time ultrasound data, to guide a biopsy procedure.

The annotated regions of the medical imaging scan may be generated by a computer-aided diagnosis system at a first location, and the at least one processor may be located at a second location, remote from the first location, the first location and the second location being linked through the communication network, wherein the communication network comprises the Internet.

### BRIEF DESCRIPTION OF THE DRAWINGS

FIG. 1 shows a process flow diagram of one embodiment of the invention; and

FIG. 2 shows a schematic representation of the system architecture.

### DESCRIPTION OF THE EMBODIMENTS

The present invention will be described with respect to a process, which may be carried out through interaction with a user or automatically, to generate a composite medical image made up of MRI and ultrasonic imaging data acquired separately at a radiology center and a urology center. One skilled in the art will appreciate, however, that imaging systems of other modalities such as PET, CT, SPECT, X-ray, and the like may be used in substitution for or in conjunction with MRI and/or ultrasound to generate the composite image in accordance with this process. Further, the present invention will be described with respect to the acquisition and imaging of data from the prostate region of a patient. One skilled in the art will appreciate, however, that the present invention is equivalently applicable with data acquisition and imaging of other anatomical regions of a patient.

The medical diagnostic and treatment system and a service networked system of the current invention includes a plurality of remote medical centers, such as a radiology center and a urology center, which may include a medical treatment facility, hospital, clinic, or mobile imaging facility. There is no limit to the number of medical centers which can be included. In a preferred embodiment there is a radiology center and a urology center, which will be more fully explained hereinafter.

The medical centers may be connected to each other via a communications link. The communications link may utilize standard network technologies such as the Internet, telephone lines (e.g., T1, T3, etc. technology), wide area network, local area network, or cloud computing technology to transmit medical data between medical centers. The communications link may be a network of interconnected server nodes, which in turn may be a secure, internal, intranet, or a public communications network, such as the Internet. A private network or virtual private network is preferred, using industry standard encrypted protocols and/or encrypted files.

Such medical centers may also provide services to centralized medical diagnostic management systems, picture archiving and communications systems (PACS), teleradiology systems, etc. Such systems may be stationary or mobile, and be accessible by a known (predetermined or static) network address or a dynamically changing or alternate network addresses. As another alternative, a medical center may include a combination of such systems. Preferably, the private or virtual private network has a static network address, which helps ensure authentication of a secure communication channel. Each system is connectable and is configured to transmit data through a network and/or with at least one database.

For the purposes hereof, the systems may utilize any acceptable network, including public, open, dedicated, private, etc. The systems may also utilize any acceptable form of communications links to the network, including conventional telephone lines, fiber optics, cable modem links, digital subscriber lines, wireless data transfer systems, etc. Any known communications interface hardware and software may be utilized by the systems.

In general, a medical center may have a number of devices such as a variety of medical diagnostic and treatment systems of various modalities. The devices may include a number of networked medical image scanners connected to an internal network. Each of the network scanners may have its own workstation for individual operation and are linked together by the internal network. Further, each scanner may be linked to a local database configured to store data associated with imaging scan sessions. Each such system is provided with communications components allowing it to send and receive data over a communications link. Scanning data may be transferred to a centralized database through the communications link and a router.

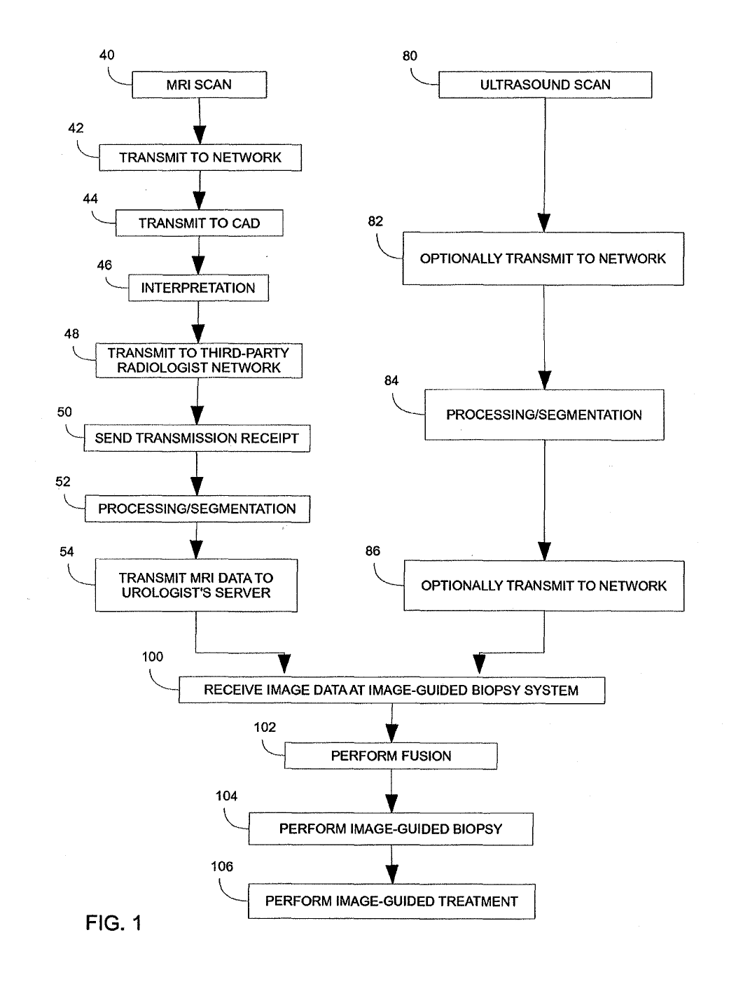

Referring now to FIG. 1, the steps of a processing technique or method for using an image-guided biopsy system for fusing MR and ultrasonic image data acquired from separate imaging systems at separate locations are set forth. The process may be guided through user interactions and commands or partially or fully automated.

The process begins with conducting one or more MRI scans 40 of a patient's prostate. Preferably, this is performed by a radiologist at a radiology center. The resulting MRI data is transmitted for storage to a network 42 of any suitable type to serve as a storage location. Network-based storage permits automated redundancy, backup and high levels of performance without burdening computing resources. The network system may include a database in which the MRI data will be stored locally within the medical center, a server at a remote location, or via cloud computing technology.

A computer assisted detection (CAD) system 44, which may include a Digital Information in Communications and Medicine (DICOM) viewer, such as DynaCAD (Invivo Corporation, Orlando, Fla.), VividLook with Versa Vue Enterprise (iCAD, Inc., Nashua, Neb.), Aegis (Hologic, Inc., Bedford, Mass.), or Segasist Prostate Auto-Contouring or Segasist Profero (Segasist Technologies, Toronto, ON, Canada), retrieves the MRI data from its storage location, through the network. It is noted that MRI data files can be quite large, and therefore a high speed network interface is preferred, such as a fiber optic interface.

The CAD system 44 may be located at any medical center, but preferably, is located at the same radiology center where the MRI scans were performed, to reduce some communication burden. Alternatively, the MRI data may be transmitted directly from the MRI equipment to the CAD system 44 via a suitable communications link. In either embodiment, the transmission of data may be carried out automatically through use of computer software, which may be hosted on a remote server or cloud computing technology.

The process continues with the interpretation 46 of the MRI scans, preferably including interpretation of at least each of the three MRI parameters. This may include identification of suspicious areas or regions of interest, and is preferably performed by a radiologist, e.g., a medical professional experienced in interpreting medical imaging data and making diagnoses and informed observations. This may be accomplished through use of the CAD system 44 and DICOM viewer. The radiologist may assess suspicious contrasts in tissue, abnormal cellular density, and unusual blood flow within the prostate. During interpretation, suspicious areas may be located on each MRI parameter and assigned a suspicion index or image grade. The region of interest may then be delineated on the axial T2-weighted images using an annotation (or annotating) tool in a DICOM reader, such as OsiriX or other software. That is, while the radiological analysis is preferably performed on a plurality of MRI parameters, these images need not be fused, and instead the resulting annotated image may be a single MRI parameter image.

Following interpretation, the resulting data, e.g., annotated radiological image, is transmitted via a communications link to, e.g., a third-party network 48, which preferably is hosted by a radiologist, who may be located at the aforementioned radiology center or at a different medical center. A transmission receipt 50, such as an electronic signal, is transmitted to the radiologist to indicate that the interpreted MRI data has been received at the third-party network.

Once received, the radiologist performs processing 52 of the MRI data, which includes segmentation. A smooth 3D model of the region of interest may then be generated. Spatial coordinates of the model may be output to a text file. In this way, a 3D model may be generated for each region of interest. A digital file containing the post-processed MRI data is generated. In general, it is preferred that regions of interest are accurately modeled, so the annotation data provides clues to the modeling process of critical physical constraints. In the more general case, the MRI model may be formulated without any annotations, and indeed the 3D modeling may be performed prior to or concurrently with the radiological analysis. However, a radiologist will typical annotate 2D slices of radiological images, which does not require a 3D model, and the 3D modeling may benefit from a focus in accurately modeling the regions of interest, and thus in a preferred embodiment, the analysis precedes the segmentation.

Thus, two distinct radiological tasks are performed; the first is a medical analysis of the medical images to determine areas of interest or suspicion for biopsy, and the second is a processing of the medical image to produce a 3D model. The former is typically performed by a trained radiologist, while the later may be performed by a skilled technician or highly automated processing center. These tasks utilize different professional expertise, and equipment, and indeed may use or exploit different data, since the 3D modeling has a different scope and purpose than the annotation.

The segmentation and/or digitizing may be carried out semi-automatically (manual control over automated image processing tasks) or automatically using computer software. One example of computer software which may be suitable includes 3D Slicer (www.slicer.org), an open source software package capable of automatic image segmentation, manual editing of images, fusion and co-registering of data using rigid and non-rigid algorithms, and tracking of devices for image-guided procedures.

See, e.g. (each of which is expressly incorporated herein by reference):

Caskey C F, Hlawitschka M, Qin S, Mahakian L M, Cardiff R D, et al. “An Open Environment CT-US Fusion for Tissue Segmentation during Interventional Guidance”, PLoS ONE 6(11): e27372. doi:10.1371/journal.pone.0027372 (Nov. 23, 2011) www.plosone.org/article/info%3Adoi%2F10.1371%2Fjoumal.pone.0027372;

Shogo Nakano, Miwa Yoshida, Kimihito Fujii, Kyoko Yorozuya, Yukako Mouri, Junko Kousaka, Takashi Fukutomi, Junko Kimura, Tsuneo Ishiguchi, Kazuko Ohno, Takao Mizumoto, and Michiko Harao, “Fusion of MRI and Sonography Image for Breast Cancer Evaluation Using Real-time Virtual Sonography with Magnetic Navigation: First Experience”, Jpn. J. Clin. Oncol. (2009) 39(9): 552-559 first published online Aug. 4, 2009 doi:10.1093/jjco/hyp087;

Porter, Brian C., et al. “Three-dimensional registration and fusion of ultrasound and MRI using major vessels as fiducial markers.” Medical Imaging, IEEE Transactions on 20.4 (2001): 354-359; Kaplan, Irving, et al. “Real time MRI-ultrasound image guided stereotactic prostate biopsy.” Magnetic resonance imaging 20.3 (2002): 295-299;

Jung, E. M., et al. “New real-time image fusion technique for characterization of tumor vascularisation and tumor perfusion of liver tumors with contrast-enhanced ultrasound, spiral CT or MRI: first results.” Clinical hemorheology and microcirculation 43.1 (2009): 57-69;

Lindseth, Frank, et al. “Multimodal image fusion in ultrasound-based neuronavigation: improving overview and interpretation by integrating preoperative MRI with intraoperative 3D ultrasound.” Computer Aided Surgery 8.2 (2003): 49-69;

Xu, Sheng, et al. “Real-time MRI-TRUS fusion for guidance of targeted prostate biopsies.” Computer Aided Surgery 13.5 (2008): 255-264;

Singh, Anurag K., et al. “Initial clinical experience with real-time transrectal ultrasonography-magnetic resonance imaging fusion-guided prostate biopsy.” BJU international 101.7 (2007): 841-845;

Pinto, Peter A., et al. “Magnetic resonance imaging/ultrasound fusion guided prostate biopsy improves cancer detection following transrectal ultrasound biopsy and correlates with multiparametric magnetic resonance imaging.” The Journal of urology 186.4 (2011): 1281-1285;

Reynier, Christophe, et al. “MRI/TRUS data fusion for prostate brachytherapy. Preliminary results.” arXiv preprint arXiv:0801.2666 (2008);

Schlaier, J. R., et al. “Image fusion of MR images and real-time ultrasonography: evaluation of fusion accuracy combining two commercial instruments, a neuronavigation system and a ultrasound system.” Acta neurochirurgica 146.3 (2004): 271-277;

Wein, Wolfgang, Barbara Röper, and Nassir Navab. “Automatic registration and fusion of ultrasound with CT for radiotherapy.” Medical Image Computing and Computer-Assisted Intervention—MICCAI 2005 (2005): 303-311;

Krücker, Jochen, et al. “Fusion of real-time transrectal ultrasound with preacquired MRI for multimodality prostate imaging.” Medical Imaging. International Society for Optics and Photonics, 2007;

Singh, Anurag K., et al. “Simultaneous integrated boost of biopsy proven, MRI defined dominant intra-prostatic lesions to 95 Gray with IMRT: early results of a phase I NCI study.” Radiat Oncol 2 (2007): 36;

Hadaschik, Boris A., et al. “A novel stereotactic prostate biopsy system integrating pre-interventional magnetic resonance imaging and live ultrasound fusion.” The Journal of urology (2011);

Narayanan, R., et al. “MRI-ultrasound registration for targeted prostate biopsy.” Biomedical Imaging: From Nano to Macro, 2009. ISBI'09. IEEE International Symposium on. IEEE, 2009;

Natarajan, Shyam, et al. “Clinical application of a 3D ultrasound-guided prostate biopsy system.” Urologic Oncology: Seminars and Original Investigations. Vol. 29. No. 3. Elsevier, 2011;

Daanen, V., et al. “MRI/TRUS data fusion for brachytherapy.” The International Journal of Medical Robotics and Computer Assisted Surgery 2.3 (2006): 256-261;

Sannazzari, G. L., et al. “CT-MRI image fusion for delineation of volumes in three-dimensional conformal radiation therapy in the treatment of localized prostate cancer.” British journal of radiology 75.895 (2002): 603-607;

Kadoury, Samuel, et al. “Realtime TRUS/MRI fusion targeted-biopsy for prostate cancer: a clinical demonstration of increased positive biopsy rates.” Prostate Cancer Imaging. Computer-Aided Diagnosis, Prognosis, and Intervention (2010): 52-62;

Comeau, Roch M., et al. “Intraoperative ultrasound for guidance and tissue shift correction in image-guided neurosurgery.” Medical Physics 27 (2000): 787;

Turkbey, Baris, et al. “Documenting the location of prostate biopsies with image fusion.” BJU international 107.1 (2010): 53-57;

Constantinos, S. P., Marios S. Pattichis, and Evangelia Micheli-Tzanakou. “Medical imaging fusion applications: An overview.” Signals, Systems and Computers, 2001. Conference Record of the Thirty-Fifth Asilomar Conference on. Vol. 2. IEEE, 2001;

Xu, Sheng, et al. “Closed-loop control in fused MR-TRUS image-guided prostate biopsy.” Medical Image Computing and Computer-Assisted Intervention—MICCAI 2007 (2007): 128-135;

Turkbey, Baris, et al. “Documenting the location of systematic transrectal ultrasound-guided prostate biopsies: correlation with multi-parametric MRI.” Cancer imaging: the official publication of the International Cancer Imaging Society 11 (2011): 31;

Tang, Annie M., et al. “Simultaneous ultrasound and MRI system for breast biopsy: compatibility assessment and demonstration in a dual modality phantom.” Medical Imaging, IEEE Transactions on 27.2 (2008): 247-254;

Wong, Alexander, and William Bishop. “Efficient least squares fusion of MRI and CT images using a phase congruency model.” Pattern Recognition Letters 29.3 (2008): 173-180;

Ewertsen, Caroline, et al. “Biopsy guided by real-time sonography fused with MRI: a phantom study.” American Journal of Roentgenology 190.6 (2008): 1671-1674;

Khoo, V. S., and D. L. Joon. “New developments in MRI for target volume delineation in radiotherapy.” British journal of radiology 79. Special Issue 1 (2006): S2-S15; and

Nakano, Shogo, et al. “Fusion of MRI and sonography image for breast cancer evaluation using real-time virtual sonography with magnetic navigation: first experience.” Japanese journal of clinical oncology 39.9 (2009): 552-559.

See also U.S. Pat. Nos. 5,227,969; 5,299,253; 5,389,101; 5,411,026; 5,447,154; 5,531,227; 5,810,007; 6,200,255; 6,256,529; 6,325,758; 6,327,490; 6,360,116; 6,405,072; 6,512,942; 6,539,247; 6,561,980; 6,662,036; 6,694,170; 6,996,430; 7,079,132; 7,085,400; 7,171,255; 7,187,800; 7,201,715; 7,251,352; 7,266,176; 7,313,430; 7,379,769; 7,438,685; 7,520,856; 7,582,461; 7,619,059; 7,634,304; 7,658,714; 7,662,097; 7,672,705; 7,727,752; 7,729,744; 7,804,989; 7,831,082; 7,831,293; 7,850,456; 7,850,626; 7,856,130; 7,925,328; 7,942,829; 8,000,442; 8,016,757; 8,027,712; 8,050,736; 8,052,604; 8,057,391; 8,064,664; 8,067,536; 8,068,650; 8,077,936; 8,090,429; 8,111,892; 8,180,020; 8,135,198; 8,137,274; 8,137,279; 8,167,805; 8,175,350; 8,187,270; 8,189,738; 8,197,409; 8,206,299; 8,211,017; 8,216,161; 8,249,317; 8,275,182; 8,277,379; 8,277,398; 8,295,912; 8,298,147; 8,320,653; 8,337,434; and US Patent Application No. 2011/0178389, each of which is expressly incorporated herein by reference.

The MRI data, which may include post-segmented MR image data, pre-segmented interpreted MRI data, the original MRI scans, suspicion index data, and/or a downloadable file containing instructions for use (described below), is transmitted via the third-party network to a server 54 controlled by a urologist, with such server being located at or connected to a network hosted by the urology center. The MRI data may be stored in a DICOM format, in another industry-standard format, or in a proprietary format unique to the imaging modality or processing platform generating the medical images. Information may also be received directly from the CAD system 44 or its associated storage system.

The urology center where the MRI data is received contains an image-guided biopsy system such as the Artemis, UroStation (KOELIS, La Tronche, France), or BiopSee (MedCom GmbH, Darmstadt, Germany). Alternatively, the image-guided biopsy system may comprise hardware and/or software configured to work in conjunction with a urology center's preexisting hardware and/or software. For example, a mechanical tracking arm may be connected to a preexisting ultrasound machine, and a computer programmed with suitable software may be connected to the ultrasound machine or the arm. In this way, the equipment already found in a urology center can be adapted to serve as an image-guided biopsy system of the type described in this disclosure. A tracking arm on the system may be attached to an ultrasound probe and an ultra sound scan 80 is performed.

A two-dimensional (2-D) or 3D model of the prostate may he generated using the ultrasonic images produced by the scan, and segmentation 84 of the model may be performed. Pre-processed ultrasound image data 82 and post-processed ultrasound image data 86 may be transmitted to a network hosted by the urology center. While the radiological data is analyzed and processed by radiologists and radiological technicians, the ultrasound data is typically obtained by the urologist, and is typically not transmitted to the radiologist for analysis since it does not include highly useful diagnostic data. That is, the ultrasound contrast for tumor vs. normal tissue is low. With automated 3D and segmentation software, the modeling can be performed within the urologist network or outsourced.

Volumetry may also be performed, including geometric or planimetric volumetry. Segmentation and/or volumetry may he performed manually or automatically by the image-guided biopsy system. Preselected biopsy sites (e.g., selected by the radiologist during the analysis) may be incorporated into and displayed on the model. All of this ultrasound data generated from these processes may be electronically stored on the urology center's server via a communications link.

As described above, processing of the MRI data or ultrasound data, including segmentation and volumetry, may be carried out manually, automatically, or semi-automatically. This may be accomplished through the use of segmentation software, such as Segasist Prostate Auto-Contouring, which may be included in the image-guided biopsy system. Such software may also be used to perform various types of contour modification, including manual delineation, smoothing, rotation, translation, and edge snapping. Further, the software is capable of being trained or calibrated, in which it observes, captures, and saves the user's contouring and editing preferences over time and applies this knowledge to contour new images. This software need not be hosted locally, but rather, may be hosted on a remote server or in a cloud computing environment.

Thus, processing of MRI data need not be performed at the radiology center in which the MRI scanning, interpretation, or grading was performed. Likewise, processing of ultrasound data need not occur at the urology center in which the ultrasonic imaging was performed. The processing for either modality may be performed remotely at any medical center which is given access to the image data and the segmentation software. For example, MRI and/or ultrasound data may be accessed by a remote medical center which performs “contouring as a service.” In this way, the processing of the image data can be outsourced to a remote medical center.

At the urology center, MRI data is integrated with the image-guided biopsy system, effectively forming a single machine. This machine is connected to the urology center's server by any suitable communications link and configured to receive the MRI data, either directly transmitted from the radiology center, or after storage in the urology center system. The image-guided biopsy system is loaded with the MRI data 100 manually, or preferably, receives it automatically. Once the image-guided biopsy system contains both the MRI data and the ultrasound data, fusion 102 of the data is performed.

The fusion process may be aided by the use of the instructions included with the MRI data. The fusion process may include registration of the MR and ultrasonic images, which may include manual or automatic selection of fixed anatomical landmarks in each image modality. Such landmarks may include the base and apex of the prostatic urethra. The two images may be substantially aligned and then one image superimposed onto the other. Registration may also be performed with models of the regions of interest. These models of the regions of interest, or target areas, may also be superimposed on the digital prostate model.

The fusion process thus seeks to anatomically align the 3D models obtained by the radiological imaging, e.g., MRI, with the 3D models obtained by the ultrasound imaging, using anatomical landmarks as anchors and performing a warping of at least one of the models to confirm with the other. The radiological analysis is preserved, such that information from the analysis relevant to suspicious regions or areas of interest are conveyed to the urologist.

The fused models are then provided for use with the real-time ultrasound system, to guide the urologist in obtaining biopsy samples.

Through the use of the described methods and systems, the 3D MR image is integrated or fused with real-time ultrasonic images, based on a 3D ultrasound model obtained prior to the procedure (perhaps immediately prior). This allows the regions of interest to be viewed under real-time ultrasonic imaging so that they can be targeted during biopsy 104.

In this way, biopsy tracking and targeting using image fusion may be performed by the urologist for diagnosis and management of prostate cancer. Targeted biopsies may be more effective and efficient for revealing cancer than non-targeted, systematic biopsies. Such methods are particularly useful in diagnosing the ventral prostate gland, where malignancy may not always be detected with biopsy. The ventral prostate gland, as well as other areas of the prostate, often harbor malignancy in spite of negative biopsy. Targeted biopsy addresses this problem by providing a more accurate diagnosis method. This may be particularly true when the procedure involves the use of multimodal MRI. Additionally, targeting of the suspicious areas may reduce the need for taking multiple biopsy samples or performing saturation biopsy.

The described methods and systems may also be used to perform saturation biopsy. Saturation biopsy is a multicore biopsy procedure in which a greater number of samples are obtained from throughout the prostate than with a standard biopsy. Twenty or more samples may be obtained during saturation biopsy, and sometimes more than one hundred. This procedure may increase tumor detection in high-risk cases. However, the benefits of such a procedure are often outweighed by its drawbacks, such as the Inherent trauma to the prostate, the higher incidence of side effects, the additional use of analgesia or anesthesia, and the high cost of processing the large amount of samples. Through use of the methods and systems of the current invention, focused saturation biopsy may be performed to exploit the benefits of a saturation biopsy while minimizing the drawbacks. After target areas suspicious of tumor are identified, a physician may sample four or more cores, all from the suspected area. This procedure avoids the need for high-concentration sampling in healthy areas of the prostate. Further, this procedure will not only improve detection, but will enable one to determine the extent of the disease.

These methods and systems of the current invention also enable physicians to later revisit the suspected areas for resampling over time in order to monitor the cancer's progression. Through active surveillance, physicians can assess the seriousness of the cancer and whether further treatment would be of benefit to the patient. Since many prostate cancers do not pose serious health threats, a surveillance program may often provide a preferable alternative to radical treatment, helping patients to avoid the risk of side effects associated with treatment.

In addition to MRI-ultrasound fusion, image-guided biopsy systems such as the Artemis may also be used in accordance with the current invention for performing an improved non-targeted, systematic biopsy under 3D ultrasonic guidance. The ultrasound image data may be remotely transmitted to the urology center, as previously described, and input to the image-guided biopsy system. When using conventional, unguided, systematic biopsy, the biopsy locations are not always symmetrically distributed and may be clustered. However, by attaching the image-guided biopsy system to an ultrasound probe, non-targeted systematic biopsy may be performed under the guidance of 3D ultrasonic imaging. This may allow for more even distribution of biopsy sites and wider sampling over conventional techniques. During biopsies performed using either MRI-ultrasound fusion or 3D ultrasonic guidance, the image data may be used as a map to assist the image-guided biopsy system in navigation of the biopsy needle, as well as tracking and recording the navigation.

The process described above provides flexibility and efficiency in performing MRI-ultrasound fusion. Although the preferred embodiment described two medical centers, every step of the fusion process may be performed at a single location, or individual steps may be performed at multiple remote locations. It is also understood that the steps of the process disclosed need not be performed in the order described in the preferred embodiment and every step need not necessarily be performed.

The process described above may further include making treatment decisions and carrying out the treatment 106 of prostate cancer using the image-guided biopsy system. The current invention provides physicians with information that can help them and patients make decisions about the course of care, whether it be watchful waiting, hormone therapy, targeted thermal ablation, nerve sparing robotic surgery, or radiation therapy. While computed tomography (CT) may be used, it can overestimate prostate volume by 35%. However, CT scans may be fused with MRI data to provide more accurate prediction of the correct staging, more precise target volume identification, and improved target delineation. For example, MRI, in combination with biopsy, will enhance patient selection for focal ablation by helping to localize clinically significant tumor foci.

In this regard, the current invention facilitates the communication of MRI and ultra sound data between radiologists and urologists to enable such physicians to perform treatment procedures effectively and efficiently. Such treatment procedures may be carried through the use of the image-guided biopsy system in conjunction with MRI and/or ultrasound data that may be generated at or transmitted to the medical center where the treatment is performed. Such treatment procedures may include the use of MRI-guided prostate laser ablation, MRI-guided prostate High Intensity Focused Ultrasound (HIFU) therapy, and/or MRI-guided prostate cryoablation therapy, among others.

White ultrasound at low intensities is commonly used for diagnostic and imaging applications, it can be used at higher intensities for therapeutic applications due to its ability to interact with biological tissues both thermally and mechanically. Thus, a further embodiment of the current invention contemplates the use of HIFU for treatment of prostate cancer in conjunction with the methods and apparatus previously described. An example of a commercially available HIFU system is the Sonablate 500 by Focus Surgery, Inc. (Indianapolis, Ind.), which is a HIFU therapy device that operates under the guidance of 3D ultrasound imaging. Such treatment systems can be improved by being configured to operate under the guidance of a fused MRI-ultrasound image.

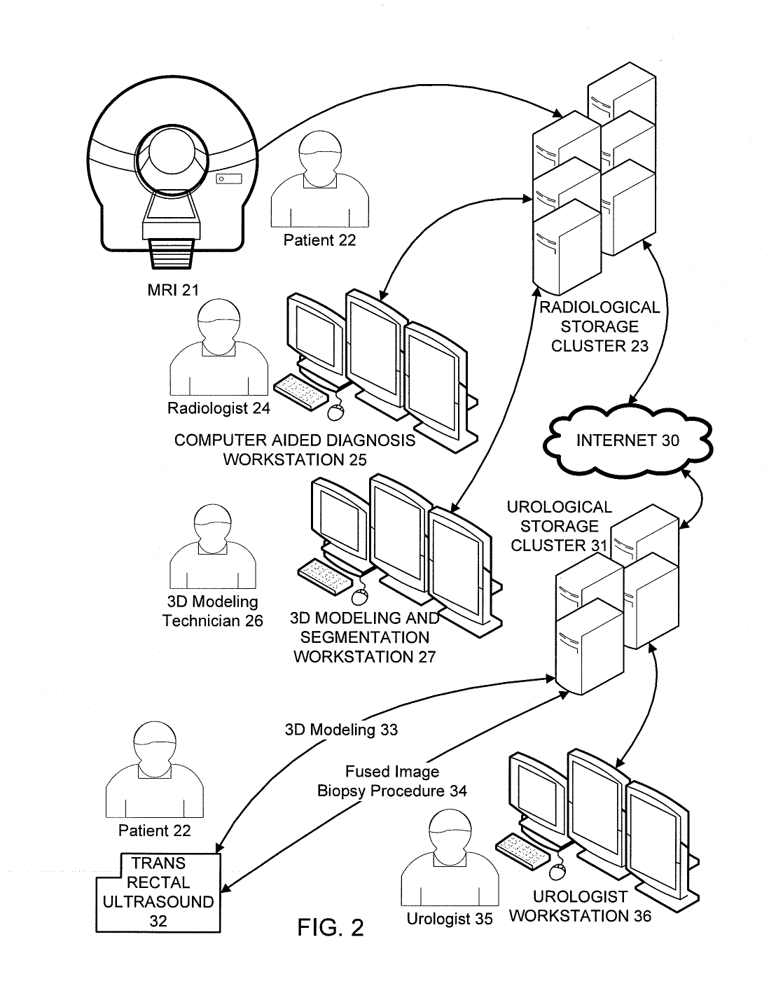

As shown in FIG. 2, a patient 22 is imaged using an MRI 21 system, with the data stored on a radiological storage cluster 23, hosted at the radiology center. A radiologist 24, with aid of a computer aided diagnosis system workstation 25, annotates the file to identify suspicious or other regions of interest. A 3D modeling technician 26, typically part of the radiology team, uses a 3D modeling and segmentation workstation to perform modeling and segmentation of the MRI images, accessing the data and/or annotated data stored on the radiological storage cluster 23. The 3D modeling technician 26 also marks the model with fixed (invariant) anatomical landmarks for subsequent registration during fusion.

The 3D model which includes the segmentation information and annotations is sent from the radiological storage cluster, through the Internet 30 to a urological storage cluster 31. At a urology center, ultrasound data is obtained using a trans rectal ultrasound 32 device, and used to generate a 3D ultrasound model, which is stored on the urological cluster 31. The ultrasound data is analyzed to identify the location of anatomical landmarks, corresponding to those identified in the 3D MRI model. The 3D MRI model is then fused with the 3D ultrasound model, either automatically or under guidance of a technician or radiologist, to form a fused model, which is also stored on the urological storage cluster. 31. The fused model preserves or is integrated with the annotations from the radiologist 23 and/or computer aided diagnosis workstation 25.

The urologist 35 then performs an invasive procedure on the patient 22, under guidance of the trans rectal ultrasound 32 system, in which the real time ultrasound data (a 2D data stream) is aligned with the fused model, showing the annotations, which represent regions which may be invisible or non-distinct on in the 2D ultrasound data alone.

In one embodiment, the image-guided biopsy system may be configured to integrate with and provide guidance to the HIFU ablation therapy equipment. In this way, rather than using the image-guided biopsy system solely for performing a diagnostic biopsy, the system may be also used in conjunction with an existing HIFU device to guide treatment of the cancer through HIFU ablation therapy.

Alternatively, the image-guided biopsy system can be configured to operate with removable and replaceable attachments for providing treatment. In this way, after performing a biopsy, the biopsy needle probe of the image-guided biopsy system may be replaced with the HIFU probe of the HIFU system.

In yet another embodiment, a specialized transducer for performing HIFU therapy is provided as an attachment to the image-guided biopsy system. This allows the image-guided biopsy system to be used not only for diagnostics, but for treatment. The current transducer used by the Artemis device is capable of imaging a full 360 degrees around the prostate as the transducer is rotated 180 degrees around the prostate, thus enabling the Artemis to generate a complete 3D image model of the prostate. However, current transducers used with HIFU therapy devices do not have such capabilities. The specialized transducer contemplated herein incorporates rotational imaging capabilities, such as those found in Artemis transducer, as well as HIFU ablation capabilities, such as those found in the Sonablate 500. Such a transducer would enable an image-guided biopsy system to perform ultrasonic imaging during HIFU ablation using a single transducer, thereby eliminating the need for removal or substitution of transducers in the patient during treatment.

Any of the above embodiments allow for HIFU ablation treatment to be performed based on fused MRI-ultrasound image-guidance. Software, located either at the medical center or on a remote server, may be used to carry out these procedures.

Alternatively, the system may be configured to perform other types of treatment, including image-guided laser ablation, radio-frequency (RF) ablation, an interstitial focal ablative therapy, or other known types of ablation therapy. The system may further be configured to perform cryoablation, brachytherapy (radiation seed placement), or other forms of cancer therapy. Such therapy may be assisted by image-guidance, such as image fusion or use of a single modality, in accordance with the current invention. Removable attachments for the image-guided biopsy system may be configured to incorporate other instrumentalities used in performing the above-listed treatment procedures.

Furthermore, during ablative therapy, temperatures in the tissue being ablated may be closely monitored and the subsequent zone of necrosis (thermal lesion) visualized. Temperature monitoring for the visualization of a treated region may reduce recurrence rates of local tumor after therapy. Techniques for the foregoing may include microwave radiometry, ultrasound, impedance tomography, MRI, monitoring shifts in diagnostic pulse-echo ultrasound, and the real-time and in vivo monitoring of the spatial distribution of heating and temperature elevation, by measuring the local propagation velocity of sound through an elemental volume of such tissue structure, or through analysis of changes in backscattered energy. Other traditional methods of monitoring tissue temperature include thermometry, such as ultrasound thermometry and the use of a thermocouple.

MRI may also be used to monitor treatment, ensure tissue destruction, and avoid overheating surrounding structures. Further, because ultrasonic imaging is not always adequate for accurately defining areas that have been treated, MRI may be used to evaluate the success of the procedure. For instance, MRI may be used for assessment of extent of necrosis shortly after therapy and for long-term surveillance for residual or recurrent tumor that may then undergo targeted biopsy.

The current invention gives physicians access to MR and ultrasonic image data and provides methods and systems to utilize such data during temperature monitoring. Removable attachments for the image-guided biopsy system may be configured to incorporate known temperature-monitoring instrumentalities.

It is further understood that imaging instrumentalities, diagnostic instrumentalities, treatment instrumentalities, such as a HIFU or laser ablation devices, temperature-monitoring instrumentalities, such as a thermocouple or ultrasound thermometry device, or any combination of such instrumentalities may be integrated into a single attachment for use with the image-guided biopsy system. Software, located either at the medical center or on a remote server, may be used to carry out these procedures.

According to another aspect of the invention, a diagnostic and treatment image generation system includes at least one database containing image data from two different modalities, such as MRI and ultrasound data, and an image-guided biopsy system. The diagnostic and treatment image generation system may also include a computer programmed to aid in the transmission of the image data and/or the fusion of the data using the image-guided biopsy system.

In accordance with yet another aspect of the present invention, a computer readable storage medium has a computer program stored thereon. The computer program represents a set of instructions that when executed by a computer cause the computer to access MRI and/or ultrasound image data of a medical patient. The computer program further causes the computer to generate an image containing the MRI data fused with the ultrasound data.

The present invention has been described in terms of the preferred embodiment, and it is recognized that equivalents, alternatives, and modifications, aside from those expressly stated, are possible and within the scope of the invention.

What is claimed is:

1. A method for guiding a procedure, comprising:
annotating regions of a medical imaging scan to acquire a first image of an organ;
modeling the medical imaging scan as an imaging scan volumetric model;
communicating the annotations of the medical imaging scan and the volumetric model through a communication network to an ultrasound center;
processing ultrasound data from an ultrasound scanner at the ultrasound center to form an ultrasound volumetric model of the organ;
fusing the medical imaging volumetric model with the ultrasound volumetric model into a fused image based on predetermined anatomical features, wherein at least one of the medical imaging volumetric model and the ultrasound volumetric model is deformed according to a tissue model such that the predetermined anatomical features of the medical imaging volumetric model and the ultrasound volumetric model are aligned; and
merging real-time ultrasound data with the fused image and annotated regions at the ultrasound center, such that that the annotated regions of the medical imaging scan are presented on a display maintaining anatomically accurate relationships with the real-time ultrasound data.

2. The method according to claim 1, wherein the modeling comprises a segmentation of anatomical features.

3. The method according to claim 1, further comprising transforming at least one of the imaging scan volumetric model and the ultrasound volumetric model to a common physical coordinate system such that the common anatomy of the organ is in a corresponding coordinate position.

4. The method according to claim 3, further comprising determining a projection of the defined features in the common physical coordinate system into a native coordinate system of the real-time ultrasound data.

5. The method according to claim 1, wherein the medical imaging scan comprises a magnetic resonance imaging scan.

6. The method according to claim 1, wherein the medical imaging scan comprises a computed aided tomography imaging scan along with co-registered PET scan.

7. The method according to claim 1, wherein the organ comprises a prostate gland.

8. The method according to claim 7, wherein the predetermined anatomical features comprise at least one portion of a urethra.

9. The method according to claim 1, wherein the medical imaging scan comprises a magnetic resonance imaging scan having plurality of magnetic resonance planar images displaced along an axis, and the ultrasound data comprises a plurality of ultrasound planar images, wherein the plurality of magnetic resonance planar images are inclines with respect to the plurality of ultrasound planar images.

10. The method according to claim 1, wherein the annotated regions are superimposed on the display of the real-time ultrasound data, to guide a biopsy procedure.

11. A system for guiding a procedure, comprising:
a memory configured to store annotated regions of a medical imaging scan of an organ;
a memory configured to store a model of the medical imaging scan as an imaging scan volumetric model;
a communication port configured to communicate the stored annotated regions and the model through a communication network;
at least one processor configured to form an ultrasound volumetric model of the organ from ultrasound data, to fuse the communicated model with the ultrasound volumetric model based on predetermined anatomical features, wherein at least one of the communicated model and the ultrasound volumetric model is deformed according to a tissue model such that the predetermined anatomical features of the communicated model and the ultrasound volumetric model are aligned; and
a real-time ultrasound system configured to merge real-time ultrasound data with the fused communicated model and ultrasound volumetric model, and to present the annotated regions on a display maintaining anatomically accurate relationships with the real-time ultrasound data.

12. The system according to claim 11, wherein the model represents a segmentation of anatomical features.

13. The system according to claim 11, further comprising at least one transform processor configured to transform at least one of the imaging scan volumetric model and the ultrasound volumetric model to a common physical coordinate system, such that the common anatomy of the organ is in a corresponding coordinate position.

14. The system according to claim 13, wherein the at least one transform processor is configured to determine a projection of the defined features in the common physical coordinate system into a native coordinate system of the real-time ultrasound data.

15. The system according to claim 11, wherein the medical imaging scan comprises a magnetic resonance imaging scan.

16. The system according to claim 11, wherein the medical imaging scan comprises a computed aided tomography imaging scan.

17. The system according to claim 11, wherein the organ comprises a prostate gland.

18. The system according to claim 17, wherein the predetermined anatomical features comprise at least one portion of a urethra.

19. The system according to claim 11, wherein the medical imaging scan comprises a magnetic resonance imaging scan having plurality of magnetic resonance planar images displaced along an axis, and the ultrasound data comprises a plurality of ultrasound planar images, wherein the plurality of magnetic resonance planar images are inclines with respect to the plurality of ultrasound planar images.

20. The system according to claim 11, wherein the annotated regions are superimposed on the display of the real-time ultrasound data, to guide a biopsy procedure.

21. The system according to claim 11, wherein the annotated regions of the medical imaging scan are generated by a computer-aided diagnosis system at a first location, and the at least one processor is at a second location, remote from the first location, the first location and the second location being linked through the communication network, wherein the communication network comprises the Internet.

22. A system for guiding a procedure, comprising:
a communication port configured to receive information defining a three dimensional volumetric model of an organ synthesized from a plurality of slices, and annotations of portions of the three dimensional volumetric model;
at least one processor configured to:
form an ultrasound volumetric model of the organ from ultrasound planar scans,
define anatomical landmarks in the ultrasound volumetric model;
define tissue deformation properties of tissues represented in the ultrasound volumetric model;
fuse the communicated three dimensional volumetric model with the ultrasound volumetric model to form a fused model, based on at least the defined anatomical features and the defined tissue deformation properties, such that the predetermined anatomical features of the three dimensional volumetric model and the ultrasound volumetric model are aligned; and
a real-time ultrasound system configured to display real-time ultrasound data with at least the annotations of the portions of the three dimensional volumetric model superimposed in anatomically accurate positions.
